(19) United States
(12) Patent Application Publication
(10) Pub. No.: US 2017/0336402 A1
(43) Pub. Date: Nov. 23, 2017

(54) BINDING ASSAY

(71) Applicant: SAPIDYNE INSTRUMENTS INC., BOISE, ID (US)

(72) Inventors: STEVE J. LACKIE, BOISE, ID (US); THOMAS R. GLASS, BOISE, ID (US)

(21) Appl. No.: 15/158,374

(22) Filed: May 18, 2016

(51) Int. Cl. G01N 33/557; G06F 19/00

(52) U.S. Cl. CPC G01N 33/557; G06F 19/706

(57) ABSTRACT

What has been developed is an improved method of determining the binding constants between two molecules that requires significantly fewer materials and potentially less time (in the case of a whole cell analysis less time to grow cell cultures as fewer cells are required in the same analysis) to undertake. The method involves utilizing an NSB measurement in preferably an n-curve analysis in order to determine the K_{d} and/or Rₜ without having to complete actual measurements to determine the lower knee of the curve(s) in the n-curve analysis. Preparing the experiment utilizing the additional samples allows an experiment represented in a binding curve having an upper and a lower knee to no longer be required to run through the lower knee to a point of completion.

In the context of analyzing receptor-ligand interaction, binding is the joining of two entities (molecules) that have some affinity for each other. This binding is almost always reversible, meaning the two molecules (generically known as ligand and receptor) will join together and come apart over and over again.

In general, these molecules associate (or bind), remain linked together for a while, and then dissociate (come apart). Eventually, an equilibrium state is reached such that the concentration of bound partners is no longer changing. Association and dissociation continue, but since the concentrations are no longer changing, the number of association events per second must equal the number of dissociation events per second.

Receptor and ligand association is important in a variety of biochemical and medical fields. Receptor and ligand association/disassociation rates play a very important role, for example, in the development of pharmaceutical drugs. The rates at which a drug interacts with its target protein in-vivo affects the duration for which the drug is active and the strength of the drug activity, and how the drug interacts with the organism's physiological system.

The disassociation constant (K_{d}) is a common way to describe the affinity between two molecules, which for the purpose of this document are called a receptor (R) and a ligand (L). K_{d} is defined as follows in Equation 1:

$${K}_{d} = \frac{[ R ] \cdot [ L ]}{[ RL ]} . \qquad Equation 1$$

Expressions representing conservation of mass are given below in Equation 2:

[Rₜₒₜₐₗ]=[R]+[RL]

[Lₜₒₜₐₗ]=[L]+[RL] Equation 2.

When Equations 1 and 2 are combined, the resulting equation provides for an equation to determine the expression for the free receptor [R] in an interaction. Alternatively the amount of bound receptor can be measured and represented as [RL] (=[Rₜₒₜₐₗ]−[R]) in the equation by substituting bound receptor for free receptor in the above and following equations:

[R]=−½·K_{d}−½·[Lₜ]+½·[Rₜ]+½·[K_{d}²+2·K_{d}·[Lₜ]+2·K_{d}·[Rₜ]+([Lₜ])²−2·[Lₜ]·[Rₜ]+([Rₜ])²]^{1/2} Equation 3.

Depending on experimental convenience, either the amount of free or bound receptor in a solution can be determined by measuring a signal (typically the voltage representing the flourescence of a tag or label applied to a molecule) that is proportional to the amount of free (or bound) receptor in the solution. This concept is expressed in the following equations in which Sig100 is the signal when the free receptor is equal to the total amount of receptor ([R]=[Rₜ]) and the non specific binding (NSB) is the signal when the receptor is fully saturated with ligand ([RL]=[Rₜ]):

$$Signal = \frac{( Sig 100 - NSB )}{[ {R}_{t} ]} \cdot [ R ] + NSB . \qquad Equation 4$$

Substituting [R] from Equation 3 into Equation 4 provides the following Equation 5:

$$Signal = \frac{( Sig 100 - NSB )}{[ {R}_{t} ]} \cdot [ \frac{- 1}{2} \cdot [ {K}_{d} + [ {L}_{t} ] - [ {R}_{t} ] - [ {K}_{d}^{2} + 2 \cdot {K}_{d} \cdot [ {L}_{t} ] + 2 \cdot {K}_{d} \cdot [ {R}_{t} ] + {( [ {L}_{t} ] )}^{2} - {2 \cdot [ {L}_{t} ] \cdot [ {R}_{t} ] + {( [ {R}_{t} ] )}^{2} ]}^{\frac{1}{2}} ] ] + NSB . \qquad Equation 5$$

Using Equation 5 in conjunction with a series of signals measured with a variety of Lt concentrations and a single Rt concentration, an analysis is performed in which the values of Sig100, NSB, K_{d} and [Rₜ] are varied iteratively to determine the best fit of the variables of the equation to the measured signals.

In FIG. 1.1, a sample set of data is provided in which known total ligand concentrations in the left column have been added to a series of identical receptor solutions. After the receptor and ligand have been allowed to come to a binding equilibrium, a signal measurement is taken that represents the free receptor concentration. The data from FIG. 1.1. is provided in a graph (FIG. 1.2) depicting the signal in volts on the Y axis representing the signal in equations 4 and 5 above plotted against the total ligand concentration [Lₜ] that was added to the receptor. As illustrated in FIG. 1.2, the data points present a general curve from which two “knees” 6, 8 are apparent.

Analysis of the data of FIGS. 1.1 and 1.2 can proceed in several alternative ways. First, the values of Lₜ can be treated as known values and values of Sig100, NSB, Rₜ, and K_{d} are determined to give a best fit of equation 5 to the data. Second, the values of Rₜ can be treated as known values and the values of Sig100, NSB, Lₜ (varied together in a fixed relationship to each other) and K_{d} are determined to give a best fit of equation 5 to the data as depicted by curve 18. Third, both Rₜ and Lₜ can be treated as known values and Sig100, NSB, and K_{d} are varied to find a best fit to the data.

Error graphs may be used to evaluate the uniqueness of fit of a parameter found as a best fit value in the procedure outlined. Error graphs are plots of the residual error (which may be expressed as a percentage of the specific binding signal) computed for a range of alternative values of a given parameter as illustrated in FIGS. 4.1-5.3. For example, the graph in FIG. 6 depicts an error curve illustrating a potential range in which the K_{d} is located within a certain confidence level. Note that the minimum in FIG. 6 occurs at the best fit value of K_{d} determined as described above and that the other three parameters (Sig100, NSB, and Rt in this case) have been reoptimized for each alternate value of K_{d} plotted.

Notice that the error graphs computed for the data of FIGS. 1.1 and 1.2, depicted in FIG. 7, show that only the K_{d} can be determined with any accuracy, whereas the Rt result only provides that the Rt is below a certain number (hence the flat portion of the line on the left side of the graph). This result is typical for a curve measured with an Rt value near to or below the K_{d} value. Conversely, in an experiment conducted at a Rt concentration far greater than the K_{d} as depicted in FIG. 8, the Rt can be resolved within a reasonable certainty but the K_{d} is only depicted as being less than a calculated number (less than 298 pM in FIG. 8).

In order to determine both the K_{d} and the Rₜ, an n-curve analysis of two or more curves is typically utilized as shown in FIG. 9. In a two curve analysis the user alters the concentration of the receptor in the second set of measurements (the receptor is fixed across each set of measurements but varied between the sets of measurements) to provide for two curves. Utilizing two curves in the n-curve analysis provides for resolution of both the Rₜ 34 and the K_{d} 36.

As discussed above several options exist in the choice of parameters to vary and parameters to hold fixed. Generally, a researcher chooses to hold fixed the parameter value that is most accurately known.

The above methodology can be applied to measuring the interaction between two molecules in which one of the molecules is located on the surface of a cell. These “whole cell measurements” are important in that the measurements are thought to more accurately represent interactions found in vivo. To perform a whole cell K_{d} measurement, typically a cell culture is grown in which the ligand is expressed on the cell surface (note: as described above ligand and receptor are generic terms and ligand on the cell may well be a cell surface receptor). A suspension of a known number of cells is typically made and subsequently a series of dilutions is performed to obtain a series of volumes of cell suspensions in which the concentration of cells is titrated. Each suspension in the series of known dilutions is then mixed with a single complimentary binding partner (receptor) concentration. The titrations are then incubated to allow the interaction between the ligand and the receptor to come to equilibrium. Typically the incubation occurs on a rocker to keep the cells in suspension. After equilibrium, the cells are centrifuged into a pellet. The remaining supernatants (containing the free receptor) are removed and measured for the concentration of receptor present. In analysis of such experiments it is most common to hold the receptor concentration (the soluble binding component) fixed and vary the effective concentration of the ligand (the cellularly expressed binding partner). This analysis choice enables calculation of the Rₜ of the ligand on the cell surface.

In general a dual curve (or any number greater than a single curve, thus an n-curve) analysis is utilized in whole cell measurements. Two or more series of cell dilutions are prepared and titrated into two or more different, known receptor concentrations. The dual (or greater) curves are typically obtained simultaneously to allow for the same cell culture to be used thus providing for the same cell Rₜ for all curves. The spacing between the multiple curves allows for further definition of the K_{d} and Rₜ by providing an additional parameter that further constrains the concentration and K_{d} variables. While one curve can be utilized in the event the cell expression (Rₜ) level is known, an n-curve analysis is typically necessary to avoid a finding of ambiguity in either the K_{d} or in the cell expression level. Further, if only one curve is initially used and the result leads to ambiguity in either the K_{d} or in the cell expression level, it may not be practical to add a second curve because the cell expression level may change from culture to culture and from time to time.

A typical whole cell n-curve analysis utilizing two curves is shown in FIG. 10. The dual curve graph charts the percent free of the receptor along the x-axis while the concentration illustrates the concentration of the cells (e.g. cells/ml) in the titrated sample. FIG. 11 constitutes the raw data from each curve depicted in FIG. 10. As shown from the data of FIG. 11, the upper knee of the curves are composed of cell dilutions 64, 70 that contain significantly fewer cells per mL than at the lower knee of the curves 66, 68. This range is from 30 million cells per mL down to 1,830 cells per mL. As illustrated by the error curves regarding the K_{d} 56 and Rₜ 58, the experiment has resolved the measurements with fair certainty.

While the experiment in FIGS. 10-11 depicts accurate data that can be obtained by utilizing a significant number of cells, in practice this approach often encounters difficulties that either limit its application or render the application unuseable. Limitations on the approach include, but are not limited to, the following: cells can be difficult, expensive, and/or time consuming to culture into large numbers; the concentration of cells needed for saturation of the receptor (the lower plateau) may be effectively solid, particularly if the cells exhibit a low expression level of the targeted ligand and/or a weak interaction between the ligand and receptor. These factors often limit the applicability of the experiment as researchers may not be inclined to invest the time and monetary resources into an experiment that may not have a clear resolution.

FIG. 12 depicts a whole cell dual curve analysis in which the data from the second knee of the curve has not been analyzed in determining the K_{d} and Rₜ of the receptor on the cell surface. In the depicted experimental curve the data from the lower knee of the curves is ignored (as shown in FIGS. 13.1 and 13.2 as ignored data 82, 84) to simulate a researcher not obtaining experimental data through the lower knee of the curve. The failure of the curves to resolve into two distinct knees depicts what was previously considered the necessity of obtaining measurements in the full range of the curve. The shape of the error curves depict that the K_{d} 76 and Rₜ 78 have not been resolved with reasonable certainty due to the absence of measurement to accurately obtain the lower knees of the curve. In addition to FIG. 12 and the associated data illustrated in FIGS. 13.1 and 13.2 depicting the procedure for and necessity of obtaining both knees of the curve(s) is further referenced in literature in the field. For example, prior publications have stated “[t]o obtain complete experimental curves that spanned the range between 0% and 100% free antibody signals, two-fold serial dilutions of cells were made . . . ”¹ and “[f]or example, to measure a 1 nM affinity, the starting antigen concentraiton present on cells should be in the range of hundreds of nanomolar to provide saturation and obtain the lower end of the K_{d} curve.”² ¹“High Affinity Binding Measurements of Antibodies to Cell-Surface-Expressed Antigens,” Rathanaswami et. al, 1 Anal. Biochem., (2008) 52-60.²“Measurement of the functional affinity constant of a monoclonal antibody for cell surface receptors using kinetic exclusion flourescence immunoassay,” Xie et. al, J. Immunological Methods, 304 (2005) 1-14.

### SUMMARY OF THE INVENTION

What has been developed is an improved method of determining the binding constants between two molecules that requires significantly fewer materials and potentially less time (in the case of a whole cell analysis less time to grow cell cultures as fewer cells are required in the same analysis) to undertake. The above described experiment no longer needs to be run through the full range of data values to establish both knees of higher and lower concentration of the curve(s). The method involves utilizing a saturated receptor measurement in preferably an n-curve analysis in order to determine the K_{d} and/or Rₜ without having to complete actual measurements to determine the lower knee of the curve(s) in the n-curve analysis. The method is thought to work to save time and materials (and therefore money) both in a whole cell analysis or alternatively in an n-curve analysis in which the molecules are weak binders thus requiring a high concentration of materials to determine the K_{d} and/or to save materials in the event the materials are scarce and/or expensive.

In the above description of the theory behind the invention the terms R and L are utilized to describe Receptor and Ligand. It is important to note that in the above theory the terms R and L are interchangeable for the purpose of the analysis. Because of this interchangeability, in the following description and in the claims the terms A and B are utilized to describe the molecules in the interaction, with either A or B being utilized to describe a receptor. Similarly, the terms A or B can be utilized to describe a ligand. The method detailed below is for determining binding constants for a molecular interaction between a first binding partner A and a second binding partner B. The first binding partner A and the second binding partner B reversibly bind to form complex AB.

The method involves a series of steps, including the step of providing two or more solutions of molecule B. In a preferred embodiment this step involves providing a plurality of first solutions having molecule B and providing a plurality of second solutions having molecule B. In a preferred embodiment the plurality of first solutions of molecule B have a uniform known concentration of molecule B across the plurality of first solutions. In a preferred embodiment the plurality of second solutions of molecule B have the same known concentration of molecule B across the plurality of second solutions. In a preferred embodiment the concentration of molecule B across the plurality of first solutions differs from the concentration of molecule B across the plurality of second solutions.

The method includes the step of providing a first NSB solution that is free of molecule B and is kept free of molecule B. This measurement can either include molecule A or be free of molecule A.

The method includes the step of adding a known amount of molecule A to the first solution and a known amount of molecule A to the second solution. In an embodiment the amount of molecule A added to the first solution is different than the known amount of molecule A added to the second solution. In a preferred embodiment this step involves adding a varying known amount of molecule A to each of first solutions in the plurality of first solutions and adding a varying known amount of molecule A to each of the second solutions in the plurality of second solutions. Alternatively molecule A can be added to the first and second solutions of molecule B sequentially or serially.

After the addition of molecule A to the solutions in the plurality of first and second solutions, the solutions are incubated, which allows for molecule A to bind to molecule B. In a preferred embodiment molecule A or molecule B is a molecule expressed on the surface of a cell. Molecule A or B can be expressed on the surface of the cell either as part of the cell's natural expression, or the cell can be engineered to express or over express molecule A or B on the surface of the cell. In an alternate embodiment in which molecule A is added to the first and second solutions of molecule B sequentially or serially, the solutions are allowed to incubate after each addition of molecule A.

After incubating the first and second solutions which contain molecule A and molecule B, the signals of free or bound molecule B in the first solutions and the second solutions are measured. In a preferred embodiment the solutions are allowed to incubate until reaching equilibrium. A measurement of the signal of free or bound molecule B in the first NSB solution is also taken. In general the measurements of free or bound can be considered as interchangeable as the computation can be made with either a direct measurement of free molecule, or with a measurement of the amount of bound molecule subtracted from the total, which would provide for the amount of free molecule in the solution.

From the measurements of the signal of free or bound molecule B in the first solutions, second solutions, and first NSB solution, the binding constants for the molecular interaction between the molecule A and the molecule B are then calculated. The binding constants are calculated, as discussed above but utilizing the measurement of the first NSB solution, by comparing a function utilizing the binding constants between molecule A and molecule B, the concentration of molecule B in the first solutions and the second solutions, the concentration of molecule A added to each of the plurality of first solutions and plurality of second solutions, the signal of the solution at 100% free or bound molecule B (Sig100), and the signal of the non specific binding (NSB) from the first solution and the second solution by varying the actual and/or theoretical values of the concentration of molecule B, Sig₁₀₀, NSB, and the binding constants to the obtained results to obtain the binding constants of best fit in the calculation, but then further utilizing the signal measured for free or bound molecule B in the first NSB solution to define the signal from solutions 1 and 2 in the presence of saturating quantity of molecule A. It is further important to note that the experimental design can vary without departing from the inventive concepts disclosed herein.

The measurement signal obtained from the first NSB solution can be utilized in several different potential mechanisms in the analysis. The measurement can be used as an actual NSB measurement in analyzing the signals from the first and second solutions. In the event that a second NSB solution is used in which the second NSB solution is free of molecule B but otherwise identical to the second solution and the first NSB solution is free of molecule B but otherwise identical to the first solution, the signal of free molecule B from the first NSB and second NSB solutions can be used as the actual NSB value for the first solution and the second solution, respectively. Additional solutions in similar concept can be added to the experimental design with the option of utilizing a further NSB solution for one or more of the additional solutions added.

Alternatively, the signal measured from the first NSB solution can be utilized as a data point to compare to the theoretical NSB value to determine an NSB value of best fit in calculating the binding constants. Similarly, a second NSB solution can be provided: the signal from the first NSB solution can be utilized as a data point to compare to the theoretical NSB value to determine an NSB value of best fit for the first solution, the signal measured from the second NSB solution would be used as a data point to compare to the theoretical NSB value to determine an NSB value of best fit for the second solution.

Alternatively the signal measured for free molecule B in the first NSB solution can be used to define a lower plateau of measurement lacking free molecule B. The NSB is then calculated by assigning a significantly greater concentration of molecule A to the signal from the first NSB solution than added to the solutions in the step of adding a known amount of molecule A to the first and second solutions. The assigned concentration of molecule A is made high enough to simulate a solution in which effectively 100% of molecule B is bound to molecule A.

The purpose of the Summary is to enable the public, and especially the scientists, engineers, and practitioners in the art who are not familiar with patent or legal terms or phraseology, to determine quickly from a cursory inspection, the nature and essence of the technical disclosure of the application. The Summary is neither intended to define the invention of the application, which is measured by the claims, nor is it intended to be limiting as to the scope of the invention in any way.

### BRIEF DESCRIPTION OF THE FIGURES

FIG. 1.1 is a table of a sample of binding signal obtained from free receptor after a series of solutions of ligand receptor of uniform concentration have been combined and allowed to equilibrate with a series of solutions with varying receptor concentration.

FIG. 1.2 is a graph of the information provided in table 1.1 FIG. 2.1 is a chart of theoretical signals and associated error calculated from the actual signal recorded in FIG. 1.1.

FIG. 2.2 is a graph of the theoretical and actual signals from FIG. 2.1.

FIG. 3.1 is a table illustrating further iterative analysis of the data presented in FIG. 2.1.

FIG. 3.2 is a table illustrating further iterative analysis of the data presented in FIG. 2.1 and resulting in a line graph presenting the lowest RMS error of the combined theoretical data.

FIG. 3.3 illustrates the results of the theoretical data with the lowest RMS error.

FIG. 4.1 illustrates the first step in plotting an error curve for the Kd data obtained in the iterative analysis described above.

FIG. 4.2 illustrates the data of the first K_{d} value plotted in FIG. 4.1.

FIG. 5.1 illustrates the actual data versus theoretical signal curve derived from the data in FIG. 3.1 but in which the K_{d} value has been varied to provide a theoretical error percent.

FIG. 5.2 illustrates the plotting of the second K_{d} value on the K_{d} error graph of FIG. 4.1.

FIG. 5.3 illustrates a second K_{d} value plotting the error graph as shown in FIG. 5.2.

FIG. 6 illustrates a K_{d} value error graph for determining a range in which the K_{d} has been determined to exist and the error associated with the values in that range.

FIG. 7 illustrates an example determination of a K_{d} for a single curve analysis and the determination of the K_{d} and Rₜ from the data depicted in the line graph. The K_{d} has been resolved with reasonable certainty while the Rₜ has not been resolved with reasonable certainty as evidenced by the lack of curvature in the left side of the graph.

FIG. 8 illustrates an example determination of a K_{d} for a single curve analysis and the determination of the K_{d} and Rₜ from the data depicted in the line graph. The Rₜ has been resolved with reasonable certainty while the K_{d} has not been resolved with reasonable certainty as evidenced by the lack of curvature in the left side of the graph.

FIG. 9 illustrates a dual curve analysis and the ability of the dual curve analysis to resolve both the Rₜ and the K_{d}.

FIG. 10 illustrates a dual curve whole cell analysis in which measurements have been obtained for data through both knees of the curve.

FIGS. 11.1-11.2 illustrate the data measurements obtained and displayed in the graphed curves of FIG. 10.

FIG. 12 illustrates a theoretical dual curve whole cell analysis in which the experiment has not run through the second knee in the curve. For illustration purposes the data are the data utilized in FIG. 10 but with data through the second knee of the curve ignored in the analysis. The error curves illustrate that neither the K_{d} nor the Rₜ of the receptor on the cell surface can be determined without obtaining a full curve.

FIGS. 13.1-13.2 illustrate the data plotted in the dual curve analysis of FIG. 12 illustrating the data from the second knee are ignored in the analysis.

FIG. 14 illustrates the dual curve whole cell analysis depicted in FIGS. 10 and 12 but in which a theoretical third solution has been measured to provide an NSB measurement utilized according to the inventive concept of this application. The use of the data point has allowed the K_{d} and Rₜ to be resolved.

FIGS. 15.1-15.2 illustrates the dual curve analysis data used in FIG. 14 but with NSB points 96, 97 utilized in the graph of FIG. 14 and associated analysis.

FIG. 16 further illustrates the dual curve analysis data of FIG. 14 but with an additional data point utilized that allows further resolution of the K_{d} and Rₜ.

FIGS. 17.1-17.2 illustrates the dual curve analysis data used in FIG. 16 but with additional actual data points 99, 101 considered in the analysis.

### DETAILED DESCRIPTION OF THE FIGURES DEPICTING THE INVENTIVE CONCEPT

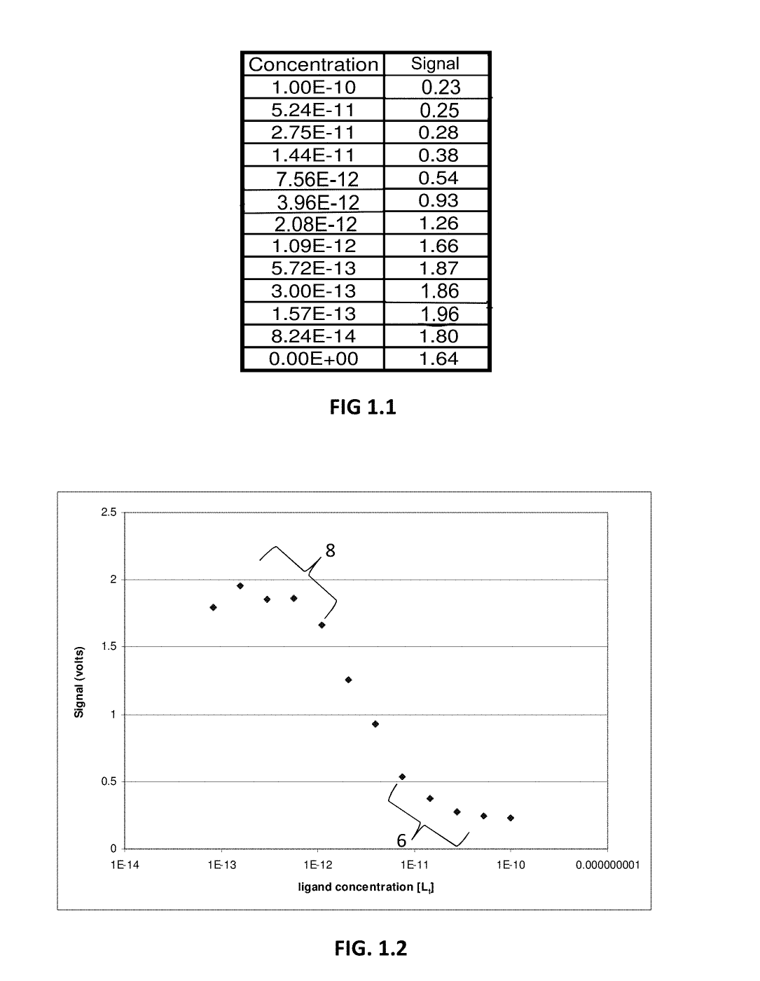

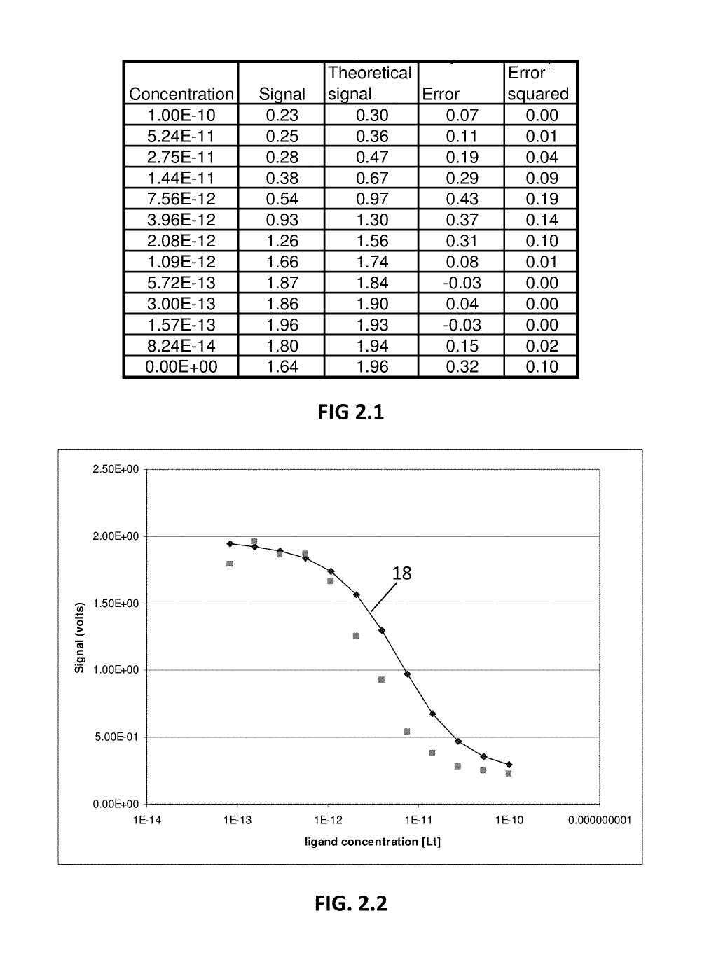

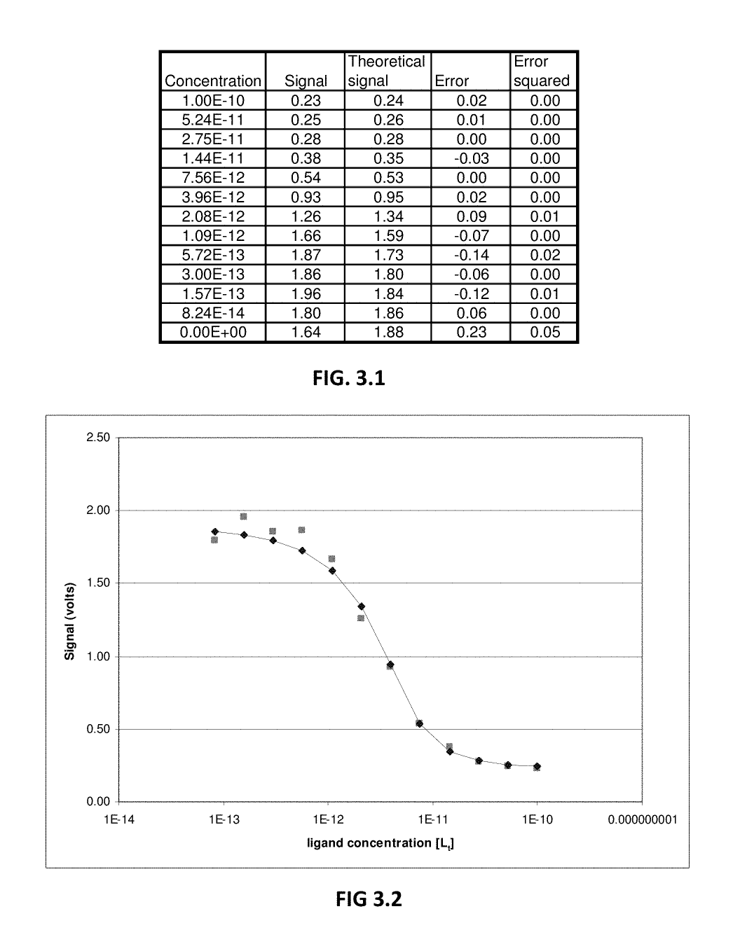

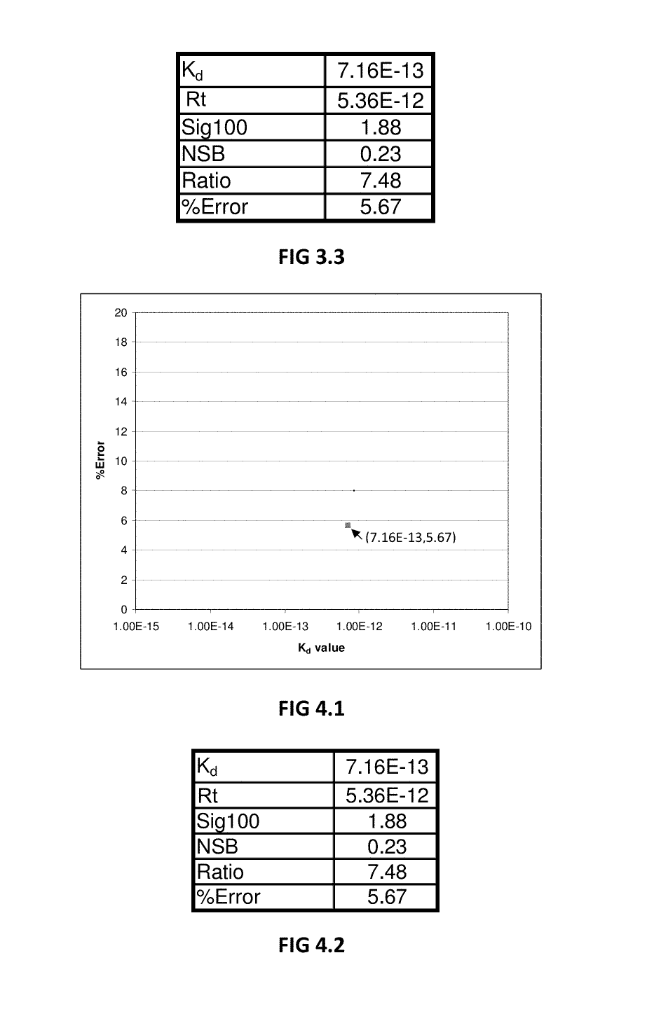

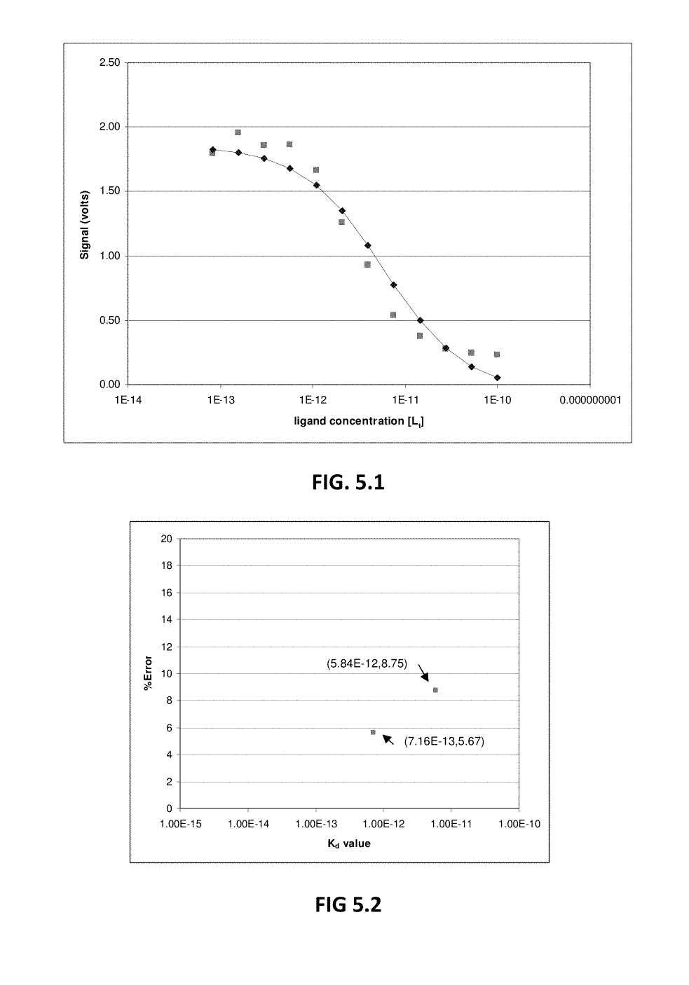

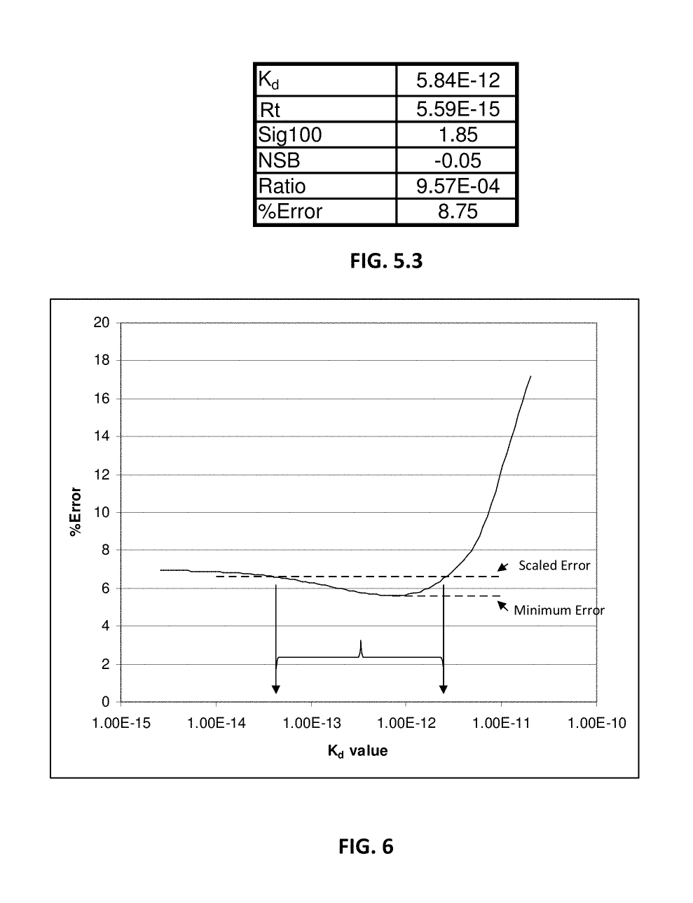

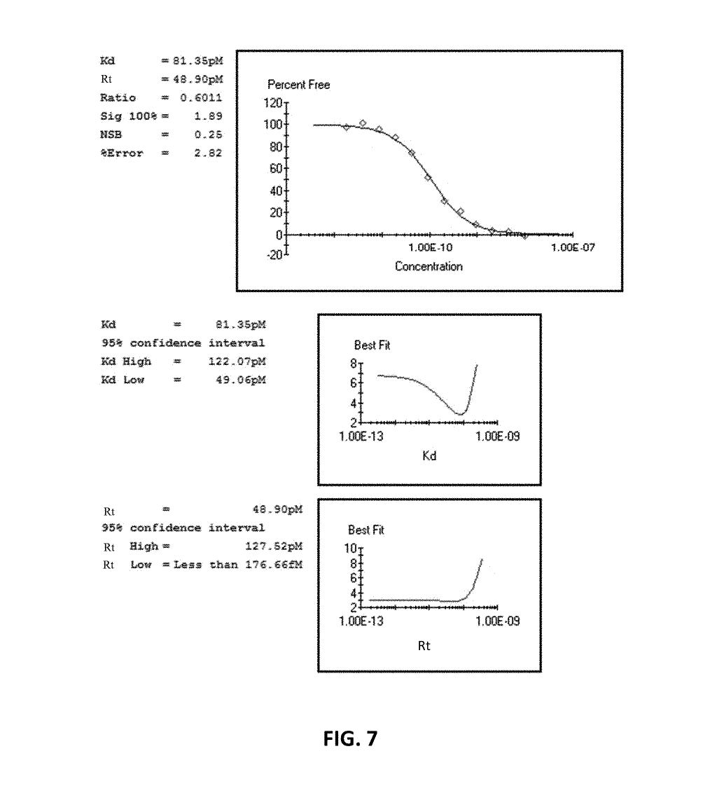

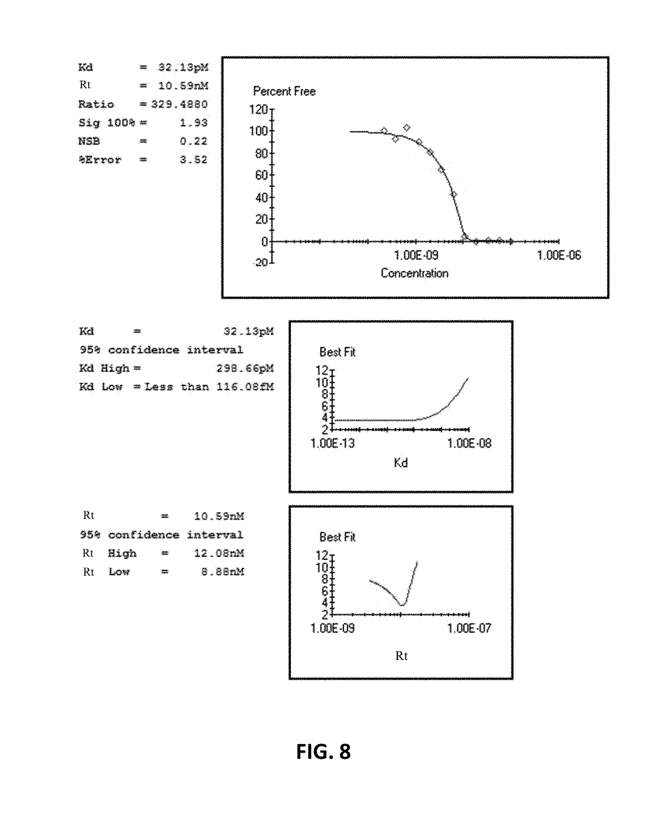

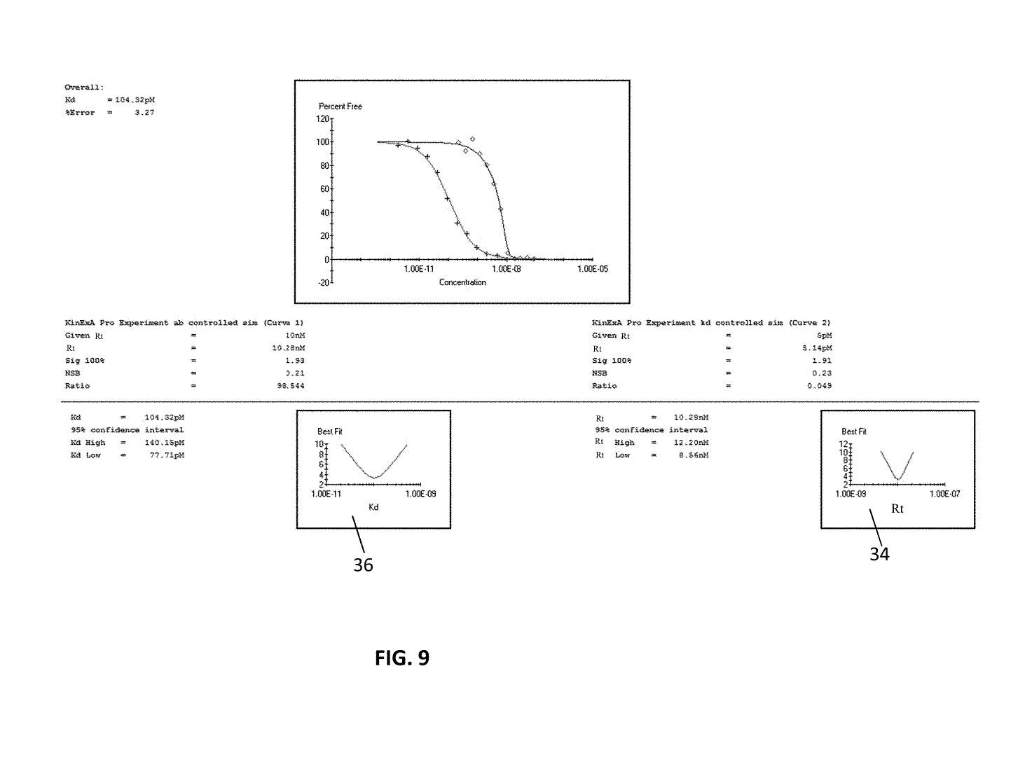

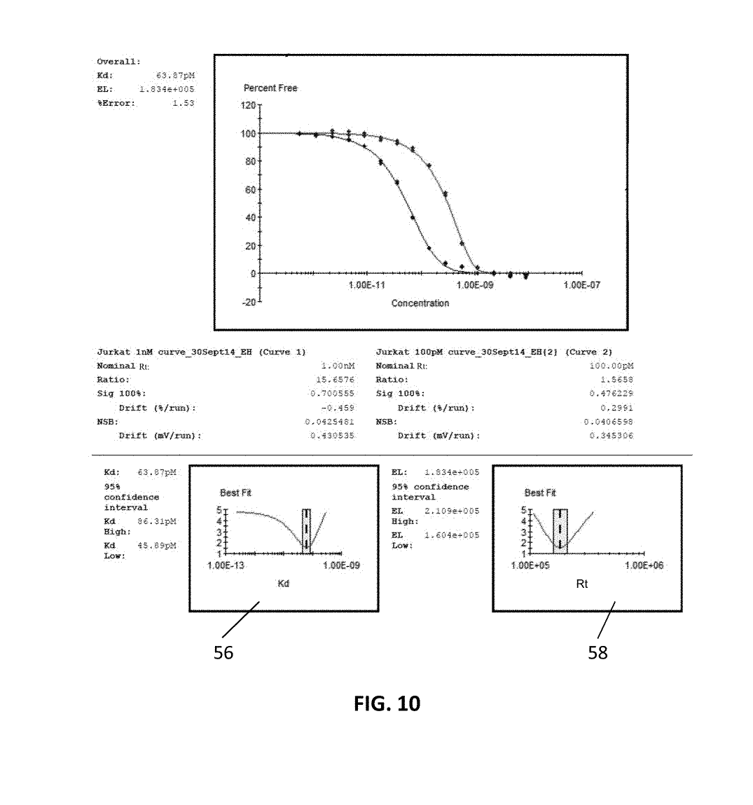

An example of the concept described in the above Background section is provided in FIGS. 1.1-9.

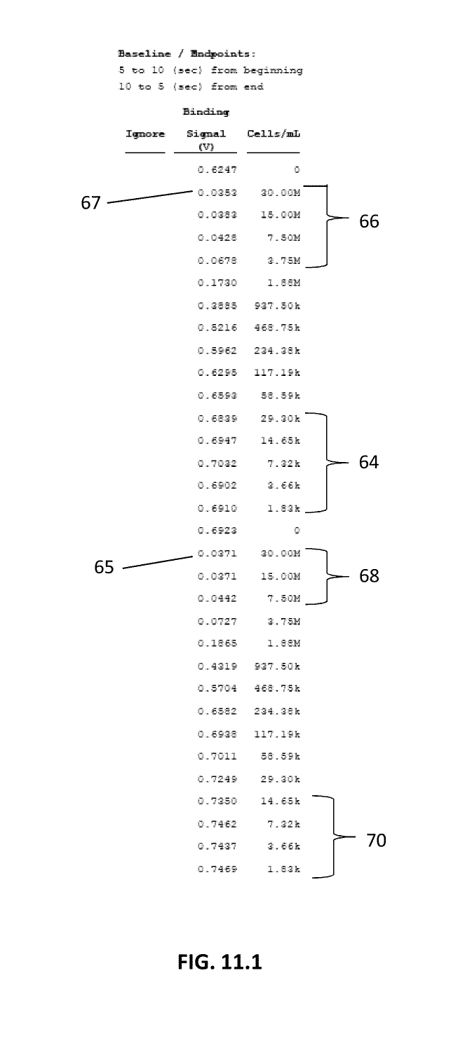

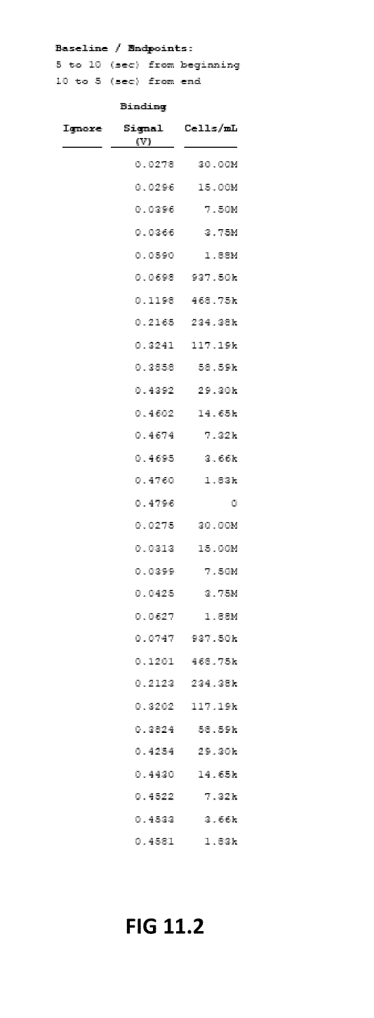

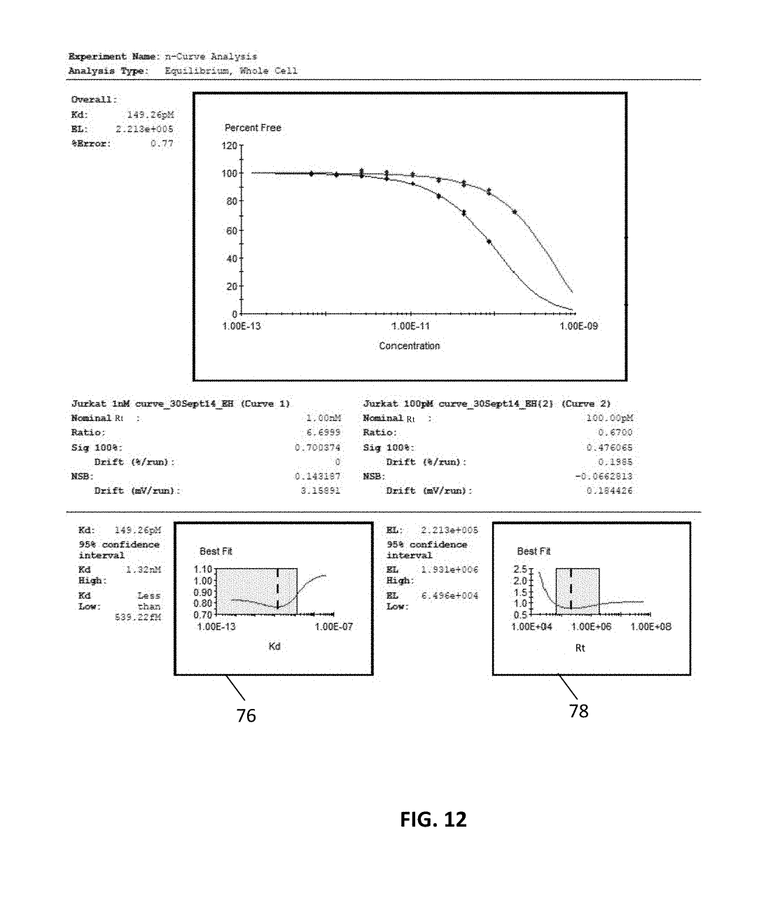

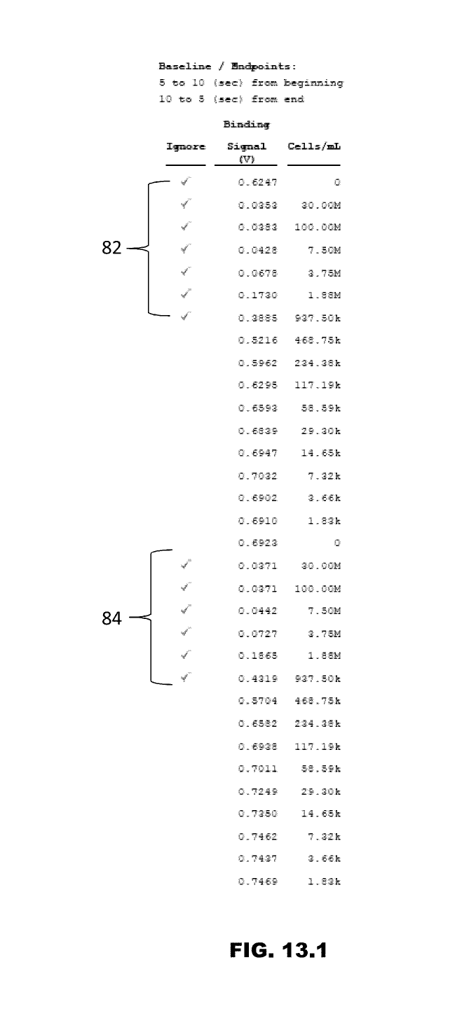

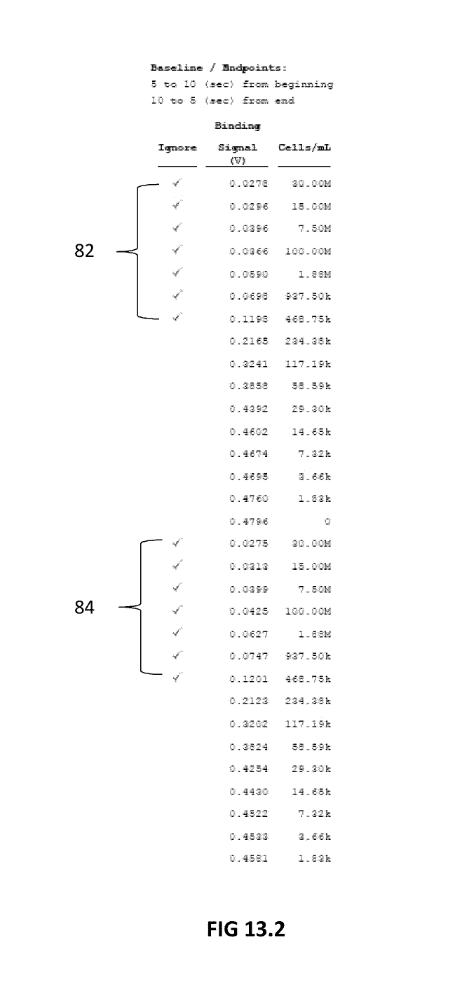

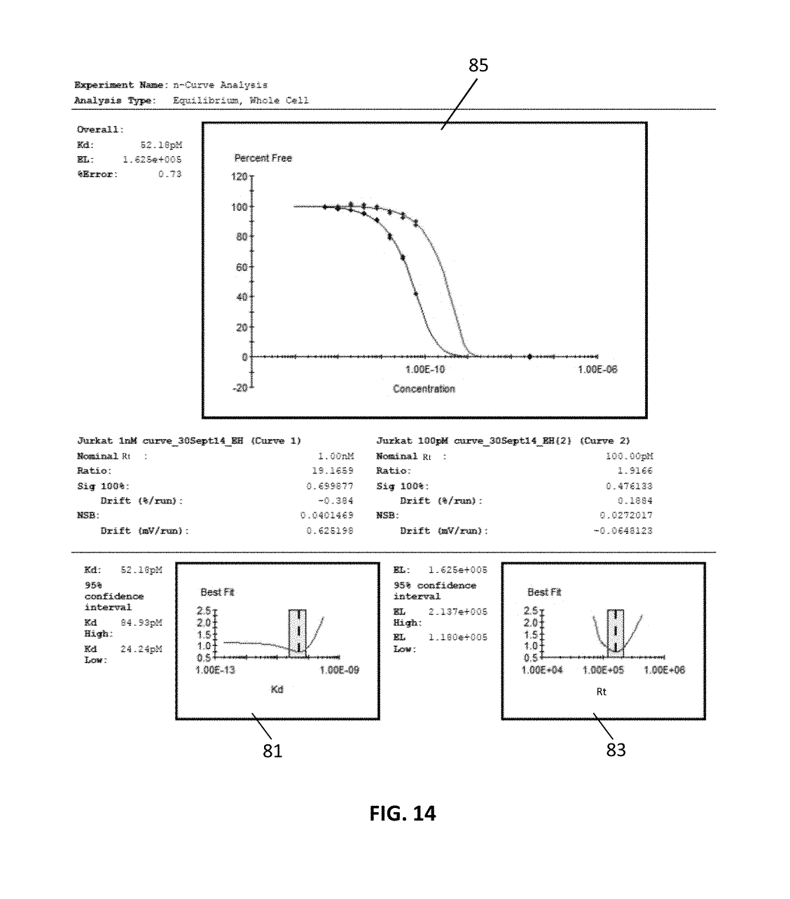

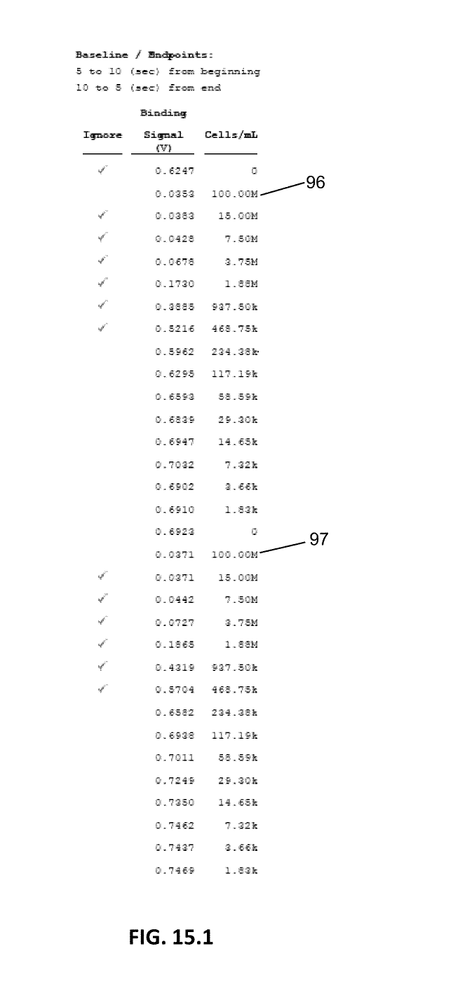

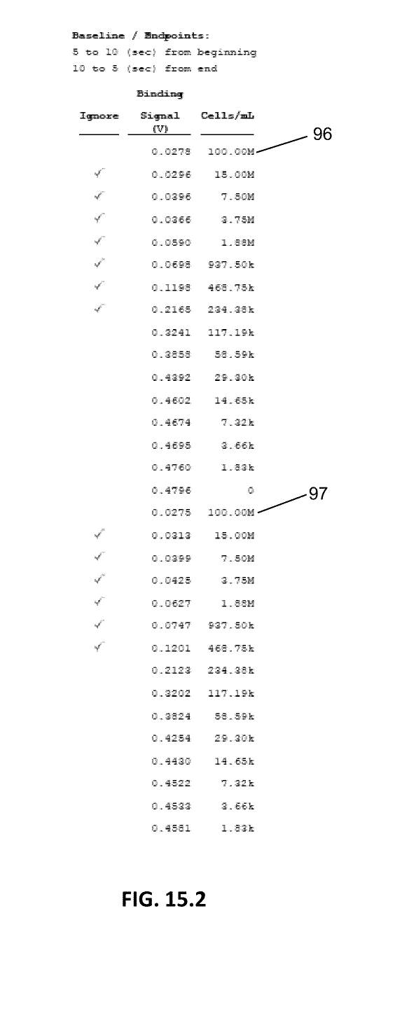

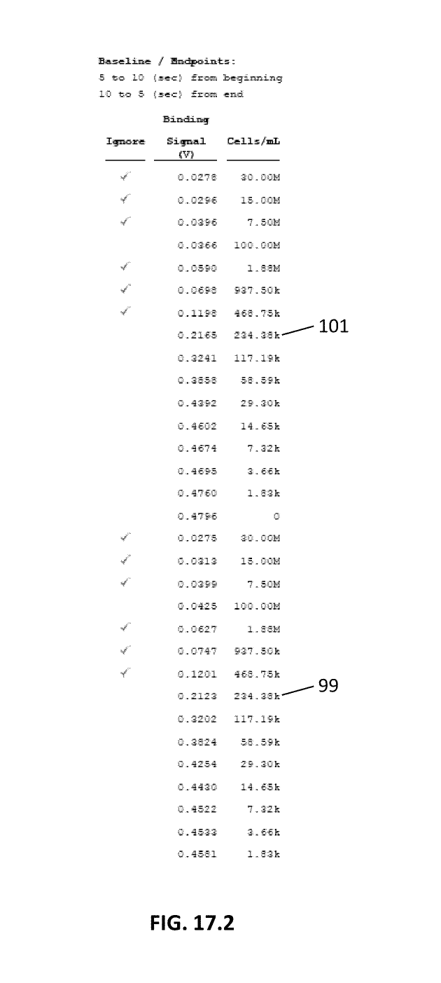

FIGS. 14-17.2 depict a whole cell dual n-curve analysis according to the above experimental description but in which the lower knee of each curve has not been determined through experimental measurement and subsequent iterative analysis as described above. Instead, an NSB measurement taken from a solution free of the receptor has been utilized in the iterative analysis described above. Utilizing the NSB measurement the analysis was able to resolve the curves 85, 86 as though actual measurements had been taken through the entire curve range and lower knee of the curve without actually having to utilize the full range of materials and time needed to conduct the experiment through these values. The analysis provided for a resolved K_{d} 81 and Rₜ 83 both with reasonable certainty and comparable to the K_{d} and Rₜ determined in FIG. 12. The benefit of this analysis is it allowed far fewer cells to be utilized in the experimental analysis. As illustrated in FIGS. 11.1 and 11.2, the experiment to determine full curve range required the experiment to proceed through a curve point having a cell concentration of 30.0M Cells/mL 65, 67. In contrast, experiments depicted in FIGS. 14-17.2 depict that it is thought that the experiment only need to run through a point to obtain the upper curve and including an NSB point in the analysis allows the analysis to resolve the full curve with a reasonable certainty.

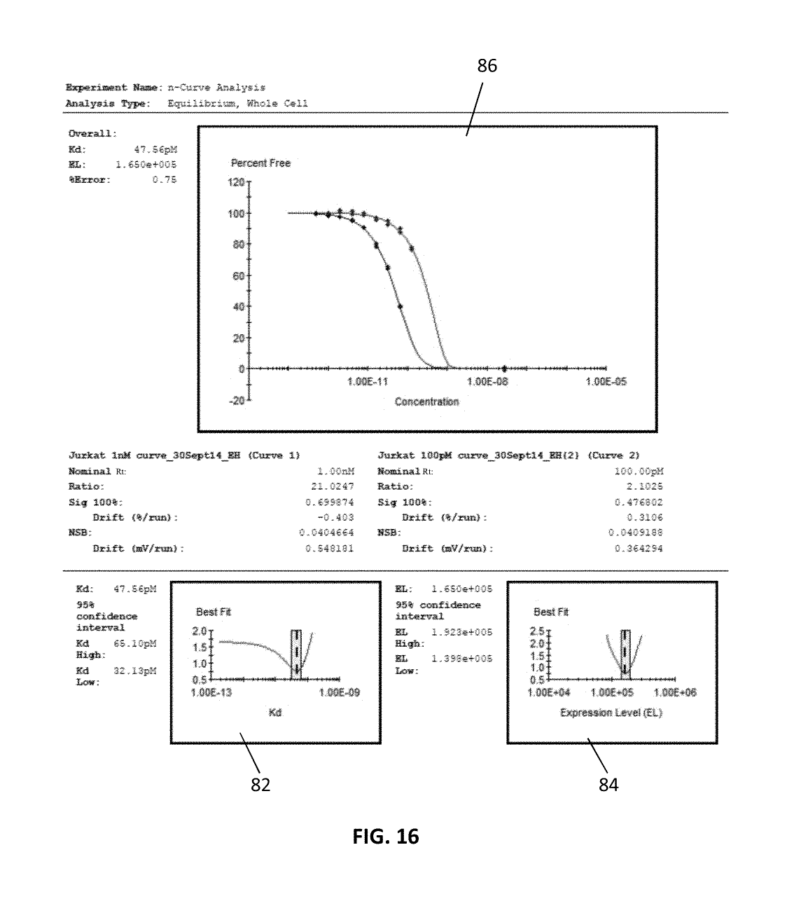

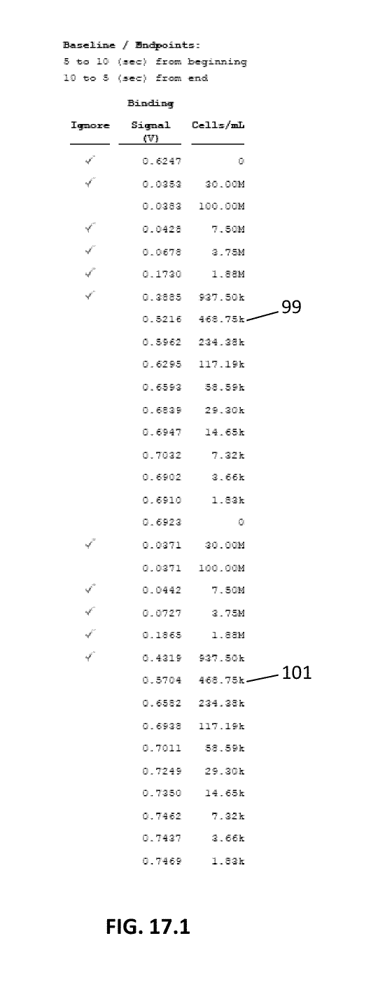

The point to which the experiment ran in FIGS. 14 and 16 went through 234.38 k cells/mL 95, 97 or 468.75 k cells/mL 99, 101. This data shows that only approximately 0.8% or 1.6%, respectively, of the total number of cells was needed to resolve the experiment by including the measurement of the first NSB solution free of molecule B (which in this case was the ligand) and still obtaining a K_{d} value 81 within reasonable certainty and Rₜ within reasonable certainty 83. This data represent a potential huge savings in time and resources required to grow a cell culture (far fewer cells can be used in the new analysis), potential large monetary savings from a researcher having to purchase far fewer cells, and/or large savings in non-whole cell analysis in which either or both of the binding molecules are expensive, precious, or both. The data further represent the ability to measure a system in which the concentrations needed to reach the second knee cannot be achieved because of the low expression level of a molecule on a cell surface and/or weak interaction between the molecules.

What is claimed is:

1. A method for determining binding constants for a molecular interaction between a first binding partner A and a second binding partner B, wherein said first binding partner and said second binding partner form complex AB, wherein said method comprises the following steps:
the step of providing at least a first solution comprising molecule B and at least a second solution comprising molecule B;
the step of providing a first NSB solution, wherein said first NSB solution is free of molecule B;
the step of adding a known amount of molecule A to said first solution and adding a known amount of molecule A to said second solution and incubating said first solutions and said second solution after adding said molecule A;
the step of measuring the signal of free molecule B in said first solution and said second solution after allowing said first solutions and said second solutions to incubate;
the step of measuring the signal of free molecule B in said first NSB solution; and
the step of calculating the binding constants for the molecular interaction between said molecule A and said molecule B wherein said binding constants are calculated by comparing a function comprised of the binding constants between molecule A and molecule B, the concentration of molecule B in said first solution and said second solution, the concentration of molecule A added to each of said first solution and said second solutions, the signal of the first solution and the second solution at 100% free molecule B (Sig100), and the signal of the non specific binding (NSB) from said first solution and said second solution by varying the actual and/or theoretical values of the concentration of free or bound molecule B, Sig₁₀₀, NSB, and the binding constants to the obtained results to obtain the binding constants of best fit in said calculation, wherein said step further comprises utilizing the signal measured for free or bound molecule B in said first NSB solution to define the signal from solutions 1 and 2 in the presence of saturating quantity of molecule A.

2. The method of determining binding constants for a molecular interaction between a first binding partner A and a second binding partner B of claim 1, wherein said step of providing a first solution comprising molecule B and a second solution comprising molecule B comprises providing a plurality of first solutions comprising molecule B and a plurality of second solutions comprising molecule B, wherein said plurality of first solutions of molecule B comprise the same known concentration of molecule B, wherein said plurality of second solutions comprise the same known concentrations of molecule B, wherein said concentration of molecule B is non-identical in said first solution and said second solution.

3. The method of determining binding constants for a molecular interaction between a first binding partner A and a second binding partner B of claim 2 wherein the step of adding a known amount of molecule A to said first solution and a known amount of molecule A to said second solution comprises adding varying known amounts of molecule A to each of said first solutions in said plurality of first solutions and varying known amounts of molecule A to each of said second solutions in said plurality of second solutions and incubating said plurality of first solutions and said plurality of second solutions after adding said molecule A.

4. The method of determining binding constants for a molecular interaction between a first binding partner A and a second binding partner B of claim 1, wherein said molecule A or said molecule B comprises a molecule expressed on a surface of a cell.

5. The method of determining binding constants for a molecular interaction between a first binding partner A and a second binding partner B of claim 1, wherein said step comprising incubating said first solution and said second solution comprises incubating said first solution and said second solution until the molecular interaction between molecule A and molecule B in each of said solutions reaches equilibrium.

6. The method of determining binding constants for a molecular interaction between a first binding partner A and a second binding partner B of claim 1, wherein said step of calculating the binding constants for the molecular interaction between said molecule A and said molecule B by including the signal measured for free molecule B comprises utilizing said signal of free molecule B from said first NSB solution as an actual NSB measurement in analyzing the signals from said first and second solutions.

7. The method of determining binding constants for a molecular interaction between a first binding partner A and a second binding partner B of claim 1, wherein said method comprises the step of adding a second NSB solution, wherein said second NSB solution is free of molecule B, wherein said first NSB solution and said second NSB solution are identical to said first solution and said second solution, respectively, but for said first NSB solution and said second NSB solution are free of molecule B; and
wherein said step of calculating said binding constants comprises utilizing the signal of free molecule B from said first NSB solution and said second NSB solution as the actual NSB value for the first solution and the second solution, respectively.

8. The method of determining binding constants for a molecular interaction between a first binding partner A and a second binding partner B of claim 1, wherein said step of calculating binding constants comprises utilizing said signal measured from said first NSB solution as a data point to compare to the theoretical NSB value to determine an NSB value of best fit in calculating said binding constants.

9. The method of determining binding constants for a molecular interaction between a first binding partner A and a second binding partner B of claim 8, wherein said method comprises the step of providing a second NSB solution free of molecule B, wherein said second NSB solution comprises an identical solution to said second solution but for said second NSB solution is free of molecule B, wherein said first NSB solution comprises an identical solution to said first solution but for said first NSB solution is free of molecule B, wherein said step of calculating binding constants comprises utilizing said signal measured from said first NSB solution as a data point to compare to the theoretical NSB value to determine an NSB value of best fit for said first solution, and wherein said step of calculating binding constants comprises utilizing said signal measured from said second NSB solution as a data point to compare to the theoretical NSB value to determine an NSB value of best fit for said second solution.

10. The method of determining binding constants for a molecular interaction between a first binding partner A and a second binding partner B of claim 1, wherein said step of calculating the binding constants for the molecular interaction between said molecule A and said molecule B further comprises utilizing the signal measured for free molecule B in said first NSB solution to define a lower plateau of measurement lacking free molecule B, wherein the NSB is calculated by assigning a significantly greater concentration of molecule A than was added to said solutions in said step of adding a known amount of molecule A to said first and second solutions to simulate a solution in which 100% of molecule B is bound to molecule A.

11. The method of determining binding constants for a molecular interaction between a first binding partner A and a second binding partner B of claim 10, wherein said step of providing a first NSB solution comprises providing at least a second NSB solution, wherein said step of calculating the binding constants further comprises utilizing the signal measured for free molecule B in said NSB solutions to define a lower plateau of measurement lacking free molecule B, wherein the NSB is calculated by assigning a significantly greater concentration of molecule A than was added to said solutions in said step of adding a known amount of molecule A to said first and second solutions to simulate a solution in which 100% of molecule B is bound to molecule A
